(19) United States
(12) Patent Application Publication
(10) Pub. No.: US 2026/0306016 A1
(43) Pub. Date: Oct. 8, 2026

(54) NON-INVASIVE DYSPNEA PREDICTION TOOL USING MACHINE LEARNING

(71) Applicant: THE REGENTS OF THE UNIVERSITY OF CALIFORNIA, Oakland, CA (US)

(72) Inventors: Karapet Mkrtchyan, San Dimas, CA (US); Erica Heinrich, Corona, CA (US)

(21) Appl. No.: 19/638,004

(22) Filed: Apr. 2, 2026

Related U.S. Application Data
(60) Provisional application No. 63/782,784, filed on Apr. 3, 2025.

(51) Int. Cl. A61B 5/00; A61B 5/0205; A61B 5/024; A61B 5/083; A61B 5/087; A61B 5/1455; G16H 20/40

(52) U.S. Cl. CPC A61B 5/4842; A61B 5/0205; A61B 5/14551; A61B 5/4836; A61B 5/7275; G16H 20/40; A61B 5/024; A61B 5/0836; A61B 5/087

(57) ABSTRACT

Disclosed herein is a machine learning system to predict breathlessness, dyspnea, and respiratory discomfort in a subject through a non-invasive manner. In some embodiments, the machine learning system monitors data on the subject, such as the electrocardiography (ECG), electromyography (EMG) of the inspiratory muscles, pulse oximetry (SpO₂), end-tidal and inspired partial pressures of oxygen (PO₂) and carbon dioxide (PCO₂), and ventilatory flow rate data.

### CROSS REFERENCE TO RELATED APPLICATIONS

This application claims priority to U.S. Provisional Application No. 63/782,784 entitled “NON-INVASIVE DYSPNEA PREDICTION TOOL USING MACHINE LEARNING,” filed Apr. 3, 2025, hereby expressly incorporated by reference in its entirety.

### RELATED FIELD

The present disclosure relates to a machine learning system or deep learning algorithm for predicting whether a subject has dyspnea. In some embodiments, the system can also predict the severity of dyspnea. In some embodiments, the learning system or algorithm monitors a patient's pO₂, pCO₂, SpO₂, EKG, EMG, or respiratory air flow, or any combination thereof.

### BACKGROUND

Dyspnea is defined as a “subjective experience of breathing discomfort”. This symptom is highly prevalent in patients with both acute and chronic respiratory and cardiovascular disease and can have a dramatic impact on clinical outcomes and patient quality of life. For example, dyspnea has been identified as a predictor of mortality in both critically and non-critically ill patients. Dyspnea also causes delays in extubation in mechanically ventilated patients, and is correlated with an increase in noninvasive ventilation failure, generally leading to worse outcomes. Hospitalized patients who experience severe dyspnea can also develop PTSD-like symptoms.

### SUMMARY OF SOME EMBODIMENTS

Disclosed herein is a machine learning method for the continuous monitoring of breathlessness/dyspnea. Also disclosed is a computer-implemented method for determining the likelihood of a subject having dyspnea. In some embodiments, the method comprises: measuring in the subject at least one of: BMI, gender, age, pO₂, pCO₂, SpO₂, heart rate, respiratory air flow, or any combination thereof; and assigning a severity score based on the at least one measurement; wherein the severity score exceeding a baseline value indicates the subject is likely to have dyspnea. In some embodiments, the measured pCO₂ is mean end tidal pCO₂, and/or maximum end tidal pCO₂. In some embodiments, the measured heart rate is mean heart rate and/or maximum heart rate. In some embodiments, the measured respiratory air flow is at least one of: mean total ventilation, maximum total ventilation, mean tidal volume, maximum tidal volume, respiratory rate, mean flow, maximum flow, or any combination thereof. In some embodiments, the measured SpO₂ is at least one of: mean SpO₂, maximum SpO₂, minimum SpO₂, or any combination thereof. In some embodiments, the severity score is on a scale of 1 to 10, optionally wherein the score is: 0-2 indicating no dyspnea, 3-5 indicating moderate dyspnea, and 6-10 indicating severe dyspnea. In some embodiments, at least BMI, end-tidal PCO₂, and SpO₂ are measured. In some embodiments, the at least one measurement(s) are repeated over a time period of at least about every 1, 2, 3, 4, 5, 10, 20, 30, 60, 120, or 180 minutes.

Also disclosed herein is a system for evaluating the severity of dyspnea in a subject. In some embodiments, the system comprises: a software system for measuring in the subject at least 1, 2, 3, 4, 5, 6, or 7 of: BMI, age, pO₂, pCO₂, SpO₂, heart rate, and respiratory air flow; wherein the software system assigns a severity score based on the at least one measurement; wherein a higher severity score is indicative of more severe dyspnea. In some embodiments, the system further comprises at least 1, 2, 3, 4, 5, or 6 of: an air flow meter, respiration monitor, SpO₂ sensor, oxygen gas analyzer, carbon dioxide gas analyzer, electrocardiogram, or any combination thereof. In some embodiments, the measured pCO₂ is mean end tidal pCO₂, and/or maximum end tidal pCO₂. In some embodiments, the measured heart rate is mean heart rate and/or maximum heart rate. In some embodiments, the measured respiratory air flow is at least one of: mean total ventilation, maximum total ventilation, mean tidal volume, maximum tidal volume, respiratory rate, mean flow, maximum flow, or any combination thereof. In some embodiments, the measured SpO₂ is at least one of: mean SpO₂, maximum SpO₂, minimum SpO₂, or any combination thereof. In some embodiments, the severity score is on a scale of 1 to 10, optionally wherein the score is: 0-2 indicating no dyspnea, 3-5 indicating moderate dyspnea, and 6-10 indicating severe dyspnea. In some embodiments, at least BMI, end-tidal PCO₂, and SpO₂ are measured. In some embodiments, the at least one measurements are repeated over a time period of at least every 1, 2, 3, 4, 5, 10, 20, 30, 60, 120, or 180 minutes.

Also disclosed herein is a method of monitoring and treating dyspnea in a subject. In some embodiments, the method comprises: measuring in the subject at least 1, 2, 3, 4, 5, 6, 7, or 8 of: BMI, gender, age, pO₂, pCO₂, SpO₂, heart rate, and respiratory air flow; assigning a severity score based on the at least one measurement; and administering a treatment to the subject if the severity score is above a threshold value. In some embodiments, the subject is mammalian and/or human. In some embodiments, the treatment comprises a known therapy, procedure, pharmaceutical, or any combination thereof that is effective against dyspnea. In some embodiments, the severity score is on a scale of 1 to 10, optionally wherein the score is: 0-2 indicating no dyspnea, 3-5 indicating moderate dyspnea, and 6-10 indicating severe dyspnea that requires administering treatment.

Support can also be found by way of the following enumerated embodiments:
1. A computer-implemented method for determining the likelihood of a subject having dyspnea, the method comprising:
   measuring in the subject at least 1, 2, 3, 4, 5, 6, 7, or 8 of: BMI, gender, age, pO₂, pCO₂, SpO₂, heart rate, and respiratory air flow; and
   assigning a severity score based on the at least one measurement;
   wherein the severity score exceeding a baseline value indicates the subject is likely to have dyspnea.
2. The method of embodiment 1, wherein the measured pCO₂ is mean end tidal pCO₂, and/or maximum end tidal pCO₂.
3. The method of embodiment 1 or 2, wherein the measured heart rate is mean heart rate and/or maximum heart rate.
4. The method of any one of the previous embodiments, wherein the measured respiratory air flow is at least one of: mean total ventilation, maximum total ventilation, mean tidal volume, maximum tidal volume, respiratory rate, mean flow, maximum flow, or any combination thereof.
5. The method of any one of the previous embodiments, wherein the measured SpO₂ is at least one of: mean SpO₂, maximum SpO₂, minimum SpO₂, or any combination thereof.
6. The method of any one of the previous embodiments, wherein the severity score is on a scale of 1 to 10, optionally wherein the score is: 0-2 indicating no dyspnea, 3-5 indicating moderate dyspnea, and 6-10 indicating severe dyspnea.
7. The method of any one of the previous embodiments, wherein at least BMI, end-tidal PCO₂, and SpO₂ are measured.
8. The method of any one of the previous embodiments, wherein the at least one measurements are repeated over a time period of at least every 1, 2, 3, 4, 5, 10, 20, 30, 60, 120, or 180 minutes.
9. A system for evaluating the severity of dyspnea in a subject, the system comprising:
   a software system for measuring in the subject at least 1, 2, 3, 4, 5, 6, or 7 of: BMI, age, pO₂, pCO₂, SpO₂, heart rate, and respiratory air flow;
   wherein the software system assigns a severity score based on the at least one measurement;
   wherein a higher severity score is indicative of more severe dyspnea.
10. The system of embodiment 9, wherein the system further comprises at least 1, 2, 3, 4, 5, or 6 of: an air flow meter, respiration monitor, SpO₂ sensor, oxygen gas analyzer, carbon dioxide gas analyzer, electrocardiogram, or any combination thereof.
11. The system of embodiment 9 or 10, wherein the measured pCO₂ is mean end tidal pCO₂, and/or maximum end tidal pCO₂.
12. The system of any one of embodiments 9-11, wherein the measured heart rate is mean heart rate and/or maximum heart rate.
13. The system of any one of embodiments 9-12, wherein the measured respiratory air flow is at least one of: mean total ventilation, maximum total ventilation, mean tidal volume, maximum tidal volume, respiratory rate, mean flow, maximum flow, or any combination thereof.
14. The system of any one of embodiments 9-13, wherein the measured SpO₂ is at least one of: mean SpO₂, maximum SpO₂, minimum SpO₂, or any combination thereof.
15. The system of any one of embodiments 9-14, wherein the severity score is on a scale of 1 to 10, optionally wherein the score is: 0-2 indicating no dyspnea, 3-5 indicating moderate dyspnea, and 6-10 indicating severe dyspnea.
16. The system of any one of embodiments 9-15, wherein at least BMI, end-tidal PCO₂, and SpO₂ are measured.
17. The system of any one of embodiments 9-16, wherein the at least one measurements are repeated over a time period of at least every 1, 2, 3, 4, 5, 10, 20, 30, 60, 120, or 180 minutes.
18. A method of monitoring and treating dyspnea in a subject, the method comprising:
   measuring in the subject at least 1, 2, 3, 4, 5, 6, 7, or 8 of: BMI, gender, age, pO₂, pCO₂, SpO₂, heart rate, and respiratory air flow;
   assigning a severity score based on the at least one measurement; and
   administering a treatment to the subject if the severity score is above a threshold value.
19. The method of embodiment 18, wherein the subject is mammalian and/or human.
20. The method of embodiment 18 or 19, wherein the treatment comprises a known therapy, procedure, pharmaceutical, or any combination thereof that is effective against dyspnea.
21. The method of any one of the previous embodiments, wherein the severity score is on a scale of 1 to 10, optionally wherein the score is: 0-2 indicating no dyspnea, 3-5 indicating moderate dyspnea, and 6-10 indicating severe dyspnea that requires administering treatment.

### BRIEF DESCRIPTION OF THE DRAWINGS

FIG. 1 depicts a nonlimiting example cartoon representation of the system used to collect data for the machine learning model.

FIG. 2 depicts a nonlimiting example statistical testing of accuracy and general usability of the learning model.

FIG. 3 depicts a nonlimiting example cartoon schematic of a system used to allow patients to give continuous data on their level of discomfort.

FIG. 4 depicts a nonlimiting example quantification comparing the accuracy of varying algorithms.

FIG. 5 depicts a nonlimiting example quantification of the true and false positive rates using the random forest algorithm.

FIG. 6 depicts a nonlimiting example quantification of which factors had the highest predictability for a patient experiencing dyspnea.

FIG. 7 depicts a nonlimiting example schematic for a decision tree based on data collected from patients.

FIG. 8 depicts a nonlimiting example quantification of the accuracy of physicians for predicting dyspnea in patients when RDOS were normalized to self-reported dyspnea severity scores, and when divided into a binary scoring system of identifying the presence of dyspnea.

FIG. 9 depicts a nonlimiting example cartoon graphic for the distribution of highest picked feature for indicating dyspnea in an individual, when each participant's data was individually run through the algorithm.

FIG. 10 depicts a nonlimiting example cartoon schematic of a machine learning system comprising a software that monitors several sensors taking reads on a patient, then generating a severity score.

FIG. 11 depicts a nonlimiting example cartoon schematic of a machine learning system comprising a software that monitors several sensors taking reads on a patient, then generating a severity score using an additional algorithm.

### DETAILED DESCRIPTION

Some embodiments of the present disclosure relate to a non-invasive machine learning model to predict whether a patient has dyspnea. In some embodiments, the machine learning model can use data collected from hospital patients regularly during their stay (by existing common equipment) to monitor a patient's level of breathing discomfort (shortness of breath/dyspnea).

Shortness of breath can dramatically affect a patient's outcome and is a predictor of mortality, yet data has shown it is very challenging for physicians to accurately predict dyspnea severity. Due to the nature of the condition, it is very common for physicians to underestimate shortness of breath, leading to worse outcomes for patients. With the method of the present disclosure, physicians will be provided with a tool for predicting and continuously monitoring shortness of breath, allowing health care professionals to better address patient needs. This is particularly helpful in the case of noncommunicative patients who can not self-report their discomfort. In some embodiments, the software collects the data from equipment that patients are already attached to, such as heart rate monitors, breathing equipment, etc., and giving a score (0-10) of how much breathing discomfort the patient is experiencing.

In the past decades, much progress was made in our understanding of the pathophysiology of dyspnea. “Breathing discomfort” is not a single sensation, and several qualitatively distinct sensations may contribute to the feeling of dyspnea. These include the sensation of work/effort of breathing, tightness or bronchoconstriction, and “air hunger” or unsatisfied inspiration. These sensations are driven by several neural inputs which modulate respiratory drive, including information from chemoreceptor stimulation, chest wall mechanoreceptors, and vagal C fibers. Dyspnea occurs when there is a mismatch between the expected result of central neural output and the actual perceived airflow or ventilation.

Despite the widespread presence of dyspnea and its significant impact on patient quality of life, the detection of dyspnea in a hospital setting continues to be a challenge. Current methods of detection rely heavily on subjective measures (look of fear, etc.) and observations that can be masked due to patient condition (grunting, nasal flaring, etc.). This leads to inaccurate estimates by healthcare professionals. Recent studies indicate that healthcare professionals significantly underestimate dyspnea severity in comparison to patients' self-report data, leading to a gap in needed care. It is likely that this disparity arises due to the complex, multivariate nature of dyspnea, as well as the variety of ways patients display and describe their discomfort. Furthermore, in many cases, patients may be unable to communicate their discomfort, leading to unrecognized patient suffering.

Due to this, there is an urgent need for a new clinical tool to address the disparity between patient self-reported dyspnea and the observations made by physicians. Such a tool should focus on objective, quantifiable data that are monitored continuously for patients in the ICU, allowing for a system that does not rely on human monitoring, but rather human intervention when needed to alleviate patient distress.

Previous studies which investigated the physiological correlates of dyspnea severity generally found weak associations. For example, factors such as increased respiratory rate and factors that lead to an increased load on the lungs have been correlated with dyspnea severity. Other factors such as increased arterial PCO2 independently correlate with dyspnea severity, primarily since hypercapnia is a contributor to air hunger. However, none of these physiological factors have a strong enough association with dyspnea severity to be used as a predictive tool alone, particularly in free-breathing individuals.

In this study, the inventors use a variety of physiological factors commonly monitored on patients in tertiary care facilities to generate an algorithm which uses 60-second intervals of data to monitor patterns associated with a patients' breathing discomfort and identify when a patient exceeds an acceptable level of dyspnea, requiring intervention. The inventors hypothesize that an algorithm derived from these quantitative values will outperform physicians using methods that involve more qualitative measurements.

Machine learning is an excellent solution for the monitoring of subjective symptoms which have some correlation to physiological measurements. Algorithms such as the one developed in the present disclosure can be extracted and used as a lightweight addition to existing monitoring software or as standalone with a small, low power computer.

### Terms

In the present disclosure, reference is made to the accompanying drawings, which form a part hereof. In the drawings, similar symbols typically identify similar components, unless context dictates otherwise. The illustrative embodiments described in this disclosure, including the drawings and claims, are not meant to be limiting. Some embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of ordinary skill in the art to which the disclosed subject matter belongs when read in light of the current disclosure.

The section headings used herein are for organizational purposes only and are not to be construed as limiting the subject matter described.

The articles “a” and “an” are used herein to refer to one or to more than one (for example, at least one) of the grammatical object of the article, unless the context dictates otherwise. By way of example, “an element” means one element or more than one element.

By “about” is meant a quantity, level, value, number, frequency, percentage, dimension, size, amount, weight or length that is approximately the recited value. Where it is not clear from the context what is encompassed by “about,” it will mean the value recited+/−10%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The terms “first,” “second,” and “third” used in combination with substances are intended to designate distinguishable features to similar substances and do not imply any particular order unless otherwise specified.

### Dyspnea Background

Dyspnea is the sensation of shortness of breath caused by a variety of clinical pathologies. The current methods of dyspnea identification are insufficient for the proper identification of dyspnea in a diverse patient population and can be inaccurate even in ideal scenarios. Dyspnea leads to lower patient quality of life and is correlated with patient mortality. Dyspnea severity can also change throughout the day, but increasing physician and nurse workloads by doing frequent face-to-face measurements would be inefficient and likely not address the issue. Therefore, there is an urgent need for continuous monitoring of dyspnea severity.

Many patients are unable to communicate their discomfort for reasons including sedation, language barriers, and cognitive impairments. However, these patients still experience dyspnea and sedation does not reduce the severity of shortness of breath.

Interventions for alleviating dyspnea exist. Particularly, titrating ventilator settings for individual patients. However, physicians need to be alerted to when these interventions are required and if an intervention is effective.

Other efforts in the field at predicting dyspnea have shortcomings, since most rely on facial data. Some patients will have reduced visibility of their facial expressions due to their condition (ventilator, clothing, bandages, etc.). Also, patients may be sedated or have other factors affecting facial expression which make it significantly more difficult to use this approach as opposed to the suggested non-invasive biomarkers. Furthermore, communication with patients is often limited, and sometimes impossible (such as ventilated patients). Also, preexisting methods involved collected detailed audio data to train the model, and will continue to require high quality audio collection of patients in order to rate dyspnea, which is not something hospitals are currently equipped to do. This goes in stark contrast to the present disclosure, which uses existing and extremely common medical equipment.

Pre-existing methods of monitoring dyspnea are dramatically more expensive to deploy in a hospital setting due to the need for additional, otherwise unused hardware to be incorporated (microphones, for example) and the limited patient pool which the prior art would apply to. Any individuals who are unable to communicate or are limited in their communication would not be aided by this type of model.

The method of the present disclosure is easier and less expensive to incorporate and can continue to monitor patients even when they are not actively communicating or even able to communicate.

### Non-Limiting Example Advantages of the Machine Learning System

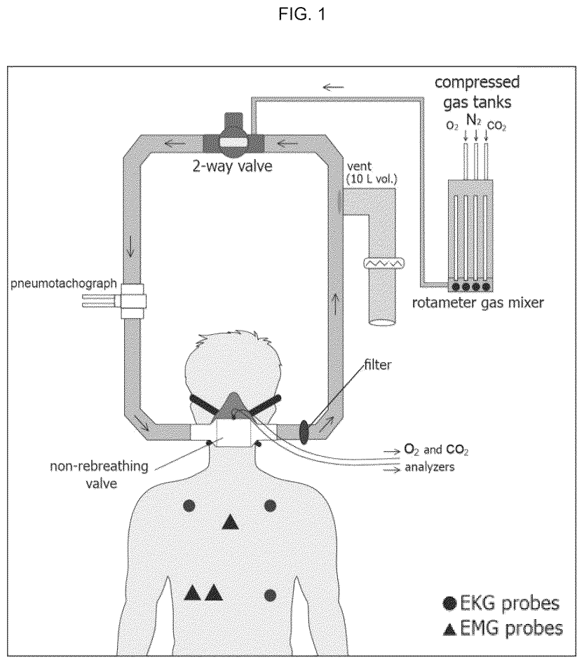

One nonlimiting example feature of the present disclosure is the ability to monitor patient breathlessness and give a score indicating the level of discomfort in a non-invasive manner. The invention (machine learning model) was developed by first gathering participants, collecting basic health data on them (sex, age, BMI, brief medical history), and using a forced end-tidal breathing system (FIG. 1) to induce dyspnea in the lab via increasing arterial carbon dioxide partial pressures or decreasing arterial oxygen pressures, both of which stimulate the sensation of dyspnea and modulate the rate of ventilation. During these bouts of dyspnea, the inventors collected electrocardiography (ECG), electromyography (EMG) of the inspiratory muscles, pulse oximetry (SpO2), end-tidal and inspired partial pressures of oxygen (PO2) and carbon dioxide (PCO2), and ventilatory flow rate data. From this data other factors can be calculated, such as heart rate, respiratory rate, and tidal volume. Throughout the experiments, participants were asked to give a score of their dyspnea in 4-minute intervals or continuously. Next, basic correlation analysis was done to determine if any of the data had trends with dyspnea severity. Some were found, including BMI, end-tidal PCO2, and SpO2. Thus, it was determined feasible to develop a machine learning algorithm to use these variables to monitor dyspnea severity. Python was used for coding, since it has well developed and accessible libraries for machine learning. The primary library used for development was scikitlearn. Supervised models were determined to be the best starting point, with the participant's self-reported dyspnea scores being used as the output, and the inputs being shown in Table 1 below:

TABLE 1

| | | | | | | | | | |
|---|---|---|---|---|---|---|---|---|---|
| | | | | | | Total | | | |
| | | Peak | | | | Vent | Total | Total | Total |
| | | PCO2 | peak | HR | HR | mean | Ventilation | volume | volume |
| Sex | BMI | Mean | PCO2 | Mean | Max | (L/min) | Max | avg | max |

| | | | | | | | | |
|---|---|---|---|---|---|---|---|---|
| | | | | | | | Peak | Peak |
| breath | Flow | Flow | SPO2 | SPO2 | SPO2 | Duration | flow | flow |
| frequency | mean | max | max | min | mean | (s) | mean | max |

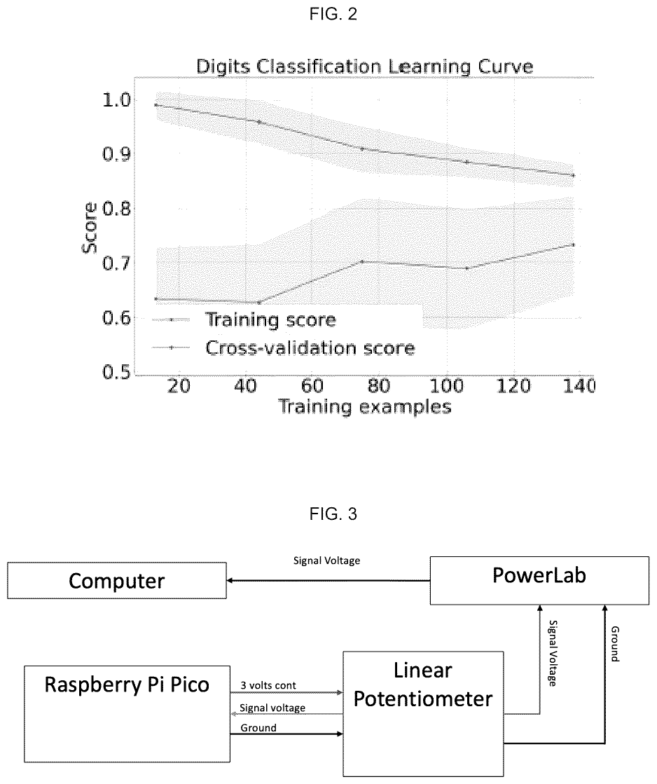

The model is then verified for accuracy (for data already present in the dataset) using cross-validation testing, as shown in FIG. 2.

In one embodiment, existing solely as software, the algorithm of the current disclosure would be integrated into existing hardware. This includes ventilators or any other patient monitoring systems used in the hospital setting. The algorithm would work in the background and display its predicted dyspnea severity score or need for intervention alongside the other medical data being shown to health care physicians. It should also be straightforward to incorporate a beeping noise or other notification when the score reaches a concerning level (these types of notifications are already extremely common on medical equipment).

However, in cases where there is no unified monitoring hardware/software in use, a piece of hardware with the software integrated can be developed to collect data from other medical devices and calculate a dyspnea score using the machine learning model.

In some embodiments, the machine learning tool of the present disclosure can be deployed onto any existing computers/monitoring hardware used by the hospital/medical center. In some embodiments, the software does not interfere with other existing monitoring equipment/software and only reads and interprets data from existing sensors. In some embodiments, the computer/monitoring system comprises some form of display or speaker to output a sound/warning for the physician/technician to indicate when it has determined that a patient requires medical intervention for their dyspnea.

### EXAMPLES

### Example 1: Initial Data Collection

Software was developed which reads monitoring data from patients and uses this information to decide whether a patient is currently dyspneic or not. The software uses sensors for pO₂, pCO₂, SpO₂, EKG, EMG, and respiratory air flow to monitor fluctuations in patient biomarkers that are relevant to dyspnea severity. In some embodiments, the software is a machine learning or deep learning algorithm.

While one nonlimiting functionality is based on the prediction model, the software of the present disclosure is also equipped to be adapted to different amounts and types of data, to provide some flexibility for different environments. Upon setup, it determines whether it is receiving all the data it expects. If there are variables missing, the software will then determine which one of its machine learning models is most suitable (outside of the default one used). If there is not enough data for any working models, it will pick one which is most suitable and factor in the missing values as part of a margin of error.

### Example 2: Sensors Used

An air flow meter is used to measure inspiratory airflow rates. The flow meter uses a differential pressure transducer to calculate air flow rates across a mesh membrane. The integral of the rate of airflow can be taken to obtain respiratory tidal volumes. The peak flow rate during inspiration is also recorded. The peak flow rate provides a measure of inspiratory effort. Both peak flow rate and tidal volume are used as input biomarkers in the predictive model.

An SpO₂ sensor is used to measure the pulse oxygen saturation. This is a measure of the proportion of total hemoglobin bound to oxygen in the arterial blood. SpO₂ is used as an input biomarker in the predictive model.

Carbon dioxide and oxygen partial pressures are measured near the mouth. Air is sampled by oxygen and carbon dioxide gas analyzers which pull air at a low flow rate. Both end-tidal partial pressure of carbon dioxide and inspired partial pressure of oxygen are input biomarkers in the predictive model.

An electrocardiogram (ECG) recording is also recorded continuously. Surface electrodes on the skin provide information about the electrical activity of the heart. From this information, heart rate is calculated as 60 divided by the interval between R waves. The heart rate and measures of heart rate variability are utilized as biomarkers in the predictive model.

As an improvement to measuring self-reported dyspnea scores at discreet intervals, a method was developed for collecting the participant's dyspnea severity scores continuously and recording it directly into the monitoring software. This was accomplished using a linear potentiometer connected to a microcontroller which sends the analog voltage signal directly to the monitoring device (PowerLab). The linear potentiometer is attached to a knob which the participant turns to indicate their level of discomfort, and the score they are selecting is shown to them at all. A schematic of this sensor is included (FIG. 3).

### Example 3: Forced End-Tidal, Semi-Rebreathing System

The system as shown in FIG. 1 comprises a loop made of respiratory tubing with an inlet to administer controlled gases (O₂, CO₂, N₂) for experimentation and a t-valve to “break” the loop and allow the participant to breath normal room air during rest periods. The inlet connects the loops to a manual gas mixer rotameter, which allows the experimenter to make the unique blend of oxygen, carbon dioxide, and nitrogen to induce the desired experimental conditions on the participant. Each of the three main channels on the rotameter are independently attached to pressurized gas tanks, and the fourth channel outputs the blended gases.

### Example 4: Development of the Machine Learning Algorithm

The algorithm was developed using the Python programming language. Sci-kit Learn was the primary library used for algorithm selection, including simple and ensembled models, preprocessing such as the label encoder, and metrics for ROC/AUC curves. Pandas was used for data frame generation, and matplotlib for visualization. To a much smaller capacity the libraries seaborn and numpy were used for additional functionality or calculations as needed.

The algorithm is trained using the biomarkers as inputs and the participant reported dyspnea scores as the output. The algorithm type used is random forest, with the following parameters:
i) 1000 equally weighted decision trees
ii) Each tree can use a maximum of five variables
iii) Random state=42 (this was mainly for reproducibility, not required)
iv) Test set=10% of data pool, training set=90%
v) Total of 10 folds

After the model was trained, it was verified using cross validation of the folds and performance metrics (including performance vs the current standard of care) were determined using confusion matrices.

### Example 5: Human Trials

Data was collected from human participants, who gave consent along with several demographic factors such as age, sex, and BMI. Participants underwent an initial health screening including lung function testing to ensure that they met study inclusion criteria. They were then fitted with the various sensors used during experiments as shown in FIG. 1. Once the sensors are fitted, the participant is fitted with an oronasal facemask connected to a semi-rebreathing circuit used for end-tidal forcing of oxygen and carbon dioxide pressures. This procedure controls end tidal and inspiratory gas tensions and thereby produces a sensation of dyspnea or “air hunger” in the participant.

The experiment is divided into six separate treatments which each participant experiences in a blinded, random order. The order is randomized using a number generator. Each treatment lasts for a total of four minutes, and rest periods of four minutes are given between each treatment. At the end of each treatment and rest period, the participant uses a handheld laser pointer at a numbered board, or turns a knob on a voltmeter, to indicate their level of respiratory discomfort.

Participant recruitment and demographics: 60 participants were recruited from winter 2023 to summer 2024. 35 women and 25 men participated with an age range of 18 to 49. The study took place at the University of California, Riverside (elevation~200 meters). Participants were recruited from the university as well as the local community through flyers and word of mouth. Inclusion criteria included individuals between the ages of 18-65 who were fluent in English, had no history of cardiovascular or pulmonary disease, were not currently taking any anti-inflammatory medications, were not currently pregnant, had no recent travel above 8,000 feet elevation within one month of the experiment, and did not smoke. Participants refrained from consuming caffeine for at least 12 hours, did not take corticosteroids, aspirin, or other anti-inflammatory medications within 48 hours of their visit, and were asked to eat a small meal prior to the start of their study appointment.

Dyspnea simulation experiments: Participants were seated in a semi-recumbent position and fitted with an oronasal face mask (7450 Series V2 Mask, Hans Rudolph, Cole Palmer, Shawnee, KS, USA) attached to the semi-rebreathing circuit via a two-way non-rebreathing valve (Model 2600, Hans Rudolph). SpO₂ was measured using a Nellcor N600x pulse oximeter (Medtronic, Minneapolis, MN, USA) with a finger probe. This pulse oximeter has been validated to accurately measure SpO₂ at levels as low as 68.5%.¹⁵ A three-lead ECG was measured using an ADInstruments (Colorado Springs CO, USA) Bioamp. Diaphragmatic EMG was also recorded using a separate Bioamp recorder, with probe placement on the seventh or eighth intercostal space at the mid-clavicular line on the right side. Partial pressures of oxygen (pO₂) and carbon dioxide (pCO₂) were subsampled from the non-rebreathing valve directly in front of the mouth. CO₂ was measured with an infrared CO₂ analyzer (Model 17630, Vacumed, Ventura, CA, USA) pulling air at a rate of 200 ml/min and O₂ was measured via a paramagnetic oxygen analyzer (Model 17620, Vacumed) pulling air at a rate of 200 ml/min. A respiratory flow head (Model MLT1000L, ADInstruments) was placed directly upstream of the inspiratory port of the non-rebreathing valve to measure inspiratory air flow rates.

Analog signals from CO₂, O₂, SpO₂, EMG, EKG, and respiratory flow measures were collected by an ADInstruments PowerLab 8/30 (model ML870) and output as a digital signal to a PC and recorded in LabChart8 pro software (AD Instruments).

To measure self-reported dyspnea severity, participants were either provided with (1) a laser pointer and were prompted at the end of each 4-minute experimental and rest treatment period to indicate their level of breathing discomfort on a large 1-10 dyspnea scale placed directly in front of them, or (2) a knob attached to a linear potentiometer which allowed them to give a continuous rating of their breathing discomfort which was recorded directly into LabChart.

To induce dyspnea, the inventors used a forced end-tidal gas method to maintain constant end-tidal PO₂ and end-tidal PCO₂ values over a four-minute period. Using a gas mixer rotameter, O₂, N₂, and CO₂ were manually adjusted to maintain these gas tensions at six pre-determined test conditions (Table 2). Each participant underwent these six trials in a randomized, participant-blinded order which each lasted four minutes, interspaced by four-minute rest periods where the participants inhaled room air.

TABLE 2
Experimental Conditions used to Collect Data

| Trial | Target Inspiratory pO2 (mmHg) | Target End-tidal pCO2 (mmHg) |
|---|---|---|
| 1 | 149 | 45 |
| 2 | 149 | 52 |
| 3 | 80 | 45 |
| 4 | 80 | 52 |
| 5 | 68 | 45 |
| 6 | 68 | 52 |

Dyspnea Severity Rating of Participants by Physicians: During the dyspnea simulation experiments participants were video recorded from a front-facing and profile view. These recordings were then divided into 30-second clips, along with screen recordings of the patients' heart rate and respiratory rate at the same time. A custom MATLAB program was created to display the clips one-by-one and prompt physicians to perform an RDOS evaluation on the patient using the given data and store their inputs for each patient/treatment. ICU Physicians and nurses, as well as Emergency department physicians completed these ratings, and their ability to assess breathlessness was determined by their RDOS scoring (above 3 points signifying the presence of dyspnea) to patients' self-reported dyspnea score. Using this detection method also allowed for an easy comparison of the physicians' performance to the machine learning algorithm. Due to difficulty observing nasal flaring with the use or an oronasal mask, this criterion was removed from the RDOS score, and total RDOS scores were therefore normalized to the total possible score for each scale when appropriate (14 for RDOS, 10 for the self-reported numeric rating scale).

### Example 6: Data Processing and Extraction

Tidal volumes were determined using the Spirometry plug in on LabChart and taking the integral of the inspiratory flow rate. Heart rate was determined as 60 divided by the R-R interval of the ECG channel. Physiological biomarker and self-reported dyspnea score data was extracted from LabChart in 1-minute increments at the end of each 4-minute trial. This allowed the participant's minute ventilation and end-tidal gas tensions to equilibrate to the treatment during the first three minutes. Averages, maximums, and minimum values were gathered for each variable. 1-minute data periods were also collected at the end of each rest cycle.

Data from the last minute of each experimental condition and rest period was averaged and used as a data point. Pre-processing included removing any 0/NA values when applicable, as well as removing data points that included physiologically impossible values (these occurred occasionally due to sensor noise, collection mistakes, etc.), or periods of unstable breathing, sighs, coughs, or speaking. Corresponding self-reported dyspnea severity scores at each timepoint were also added to the dataset. Prepared data was saved in a CSV (comma separated values) format.

Data Pre-Processing: Data was first quality checked manually to ensure all values fell within normal physiological limits. Values outside of these limits were investigated and removed row-wise if necessary. Once all the data had been verified using this method, automatic (coded) pre-processing included assigning a binary to the target variable (See generation of models section for more details on how this was done), label encoding of all non-numeric variables, and deletion of empty columns followed by deletion of rows with incomplete data.

Table 3 below shows the models which were generated originally and the parameters that were defined:

TABLE 3

| Model | Parameters |
|---|---|
| Random Forest | random state = 42 |
| Support Vector Machine | Kernal = linear, Regularization = 1 |
| K-Nearest Neighbor | Number of neighbors = 3 |
| Multinomial Logistic | Class = multinomial, solver = ‘lbfgs', |
| Regression | maximum iterations = 10,000 |

*Parameters that are not listed were left to default settings for the particular algorithm within scikitlearn.

Initial models were given input variables of sex, BMI, mean end tidal PCO₂, maximum end tidal PCO₂, mean heart rate, maximum heart rate, mean total ventilation, maximum total ventilation, mean tidal volume, maximum tidal volume, respiratory rate, mean flow, maximum flow, as well as mean, maximum and minimum SPO₂, with an output variable of dyspnea severity. Dyspnea severity was divided into three categories rather than using each measurement from 0-10 as an individual category. This was done to increase both model performance and clinical relevance of the tool; dyspnea identification was the primary goal while urgency of medical intervention was secondary. The division of categories (no dyspnea vs some dyspnea) was determined using the normalized scoring from the RDOS as well as data from exit interview with participants. The thresholds for these groups are: 0-2=no dyspnea, 3-5=moderate dyspnea, 6-10=severe dyspnea.

The random forest classifier was chosen as the primary algorithm to continue experimentation on. This is primarily for its inherent resistance to over fitment of models to test data. Since the data was collected in a small (relative to big data projects) batch of healthy participants, each individual in the dataset contributed a significant amount of data and variety. Given the vast amount of individual variation in response to the given dyspnea stimulus (see data below), over fitting the model to the collected samples was a serious consideration.

After random forest was selected as the primary model to continue developing, parameters were further adjusted. The goal of the parameter adjustments was to both increase performance and useability of the model. The maximum number of variables each decision tree could incorporate was locked to 3, and the number of estimators was set to 1000 to provide ample votes without becoming too computationally intensive. Model was given inputs of mean, maximum, minimum and standard deviation of the following variables: inspired PO₂, end tidal PCO₂, heart rate, total ventilation, tidal volume, respiratory rate, flow, and SPO₂. Additionally, biological sex and BMI were included as demographic variables, with the label encoding function in scikitlearn being implemented to turn convert the string data biological sex into a numerical binary. For the output variable, the categorization was reduced compared to the previous models to a dichotomy via the elimination of the “severe dyspnea” category. In this way, the model is performing its categorization with a similar threshold to physicians using the RDOS; as in, determining whether the individual requires medical intervention for their respiratory discomfort.

Testing of Model Performance: Models were tested for performance using k-fold cross-validation testing. The original algorithms were divided into 5 folds, each using 80% of the available data as training data and 20% as testing data. Later random forests were tested using 10 folds, each using 90% of data for training and 10% testing data. Confusion matrices were generated from the test data to calculate sensitivity and specificity.

### Example 7: Experimental Results

Some participants were unable to complete the experimental protocol for the following reasons: voluntary exiting the experiment early before completing all 6 treatments, or approaching pre-determined parameter limits (such as oxygen saturation of 70%, or minute ventilation of 100 L/min). Some experiments were removed due to errors with sensors and other data collection malfunctions. The final dataset was 60 participants with a small fraction missing data for a few treatments.

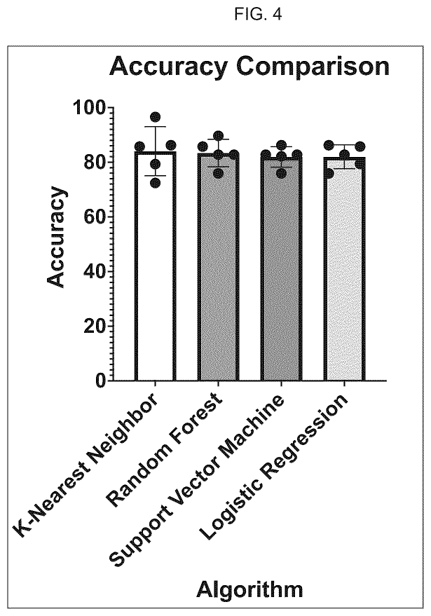

Four classification models were chosen for the dataset without any adjustment from their stock parameters in sci-kit learn to get a baseline for model performance (FIG. 4). All models and scored approximately the same in accuracy and were statistically insignificant from one another (p=0.9245). Due to their robustness to overfitting, the random forest classifier was chosen and used for the remainder of the study. The number of estimators was increased to n=1000, and a maximum feature selection was limited to 3 per decision tree. A random state of 42 was arbitrarily selected for data reproducibility.

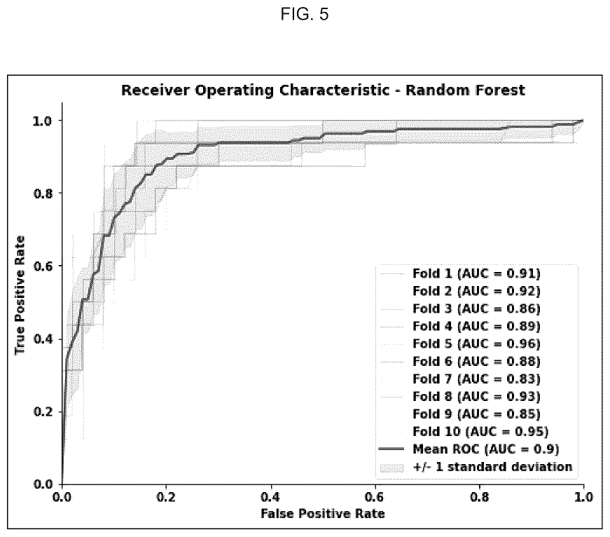

False and true positive rates were also quantified (FIG. 5). AUC scores within the range of 0.83-0.96 by creating splits with 10% testing data and 90% training data. Average AUC was 0.9, with significant diminishing returns beyond a false positive rate of about 20% (0.2).

### Example 8: Feature Importance

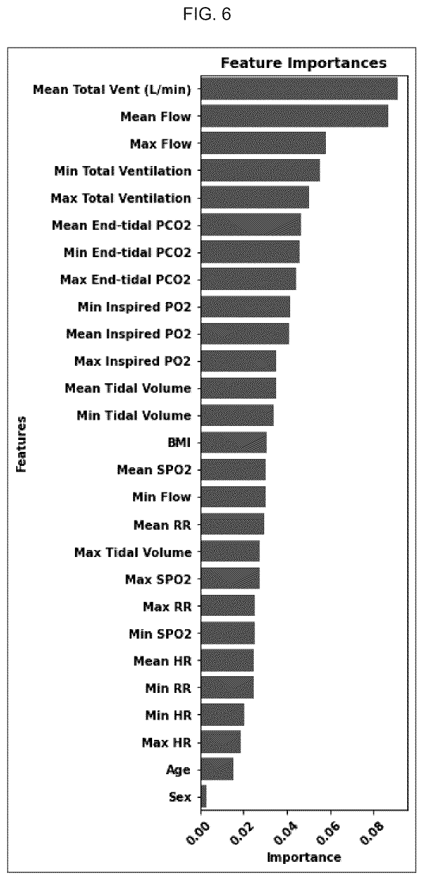

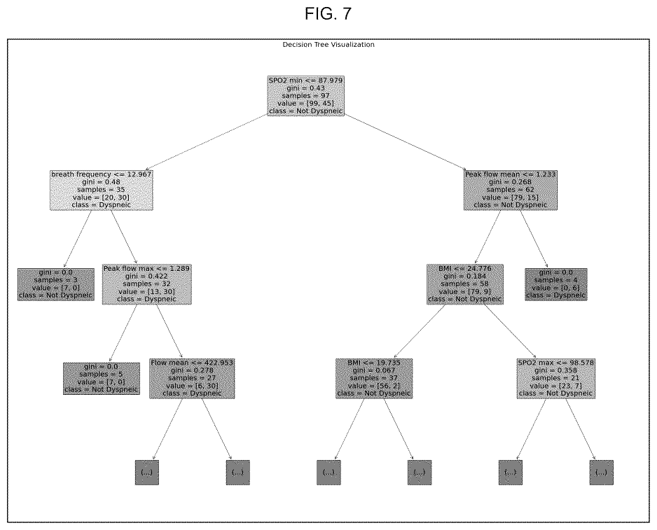

The importance of each factor in overall predictably was quantified (FIG. 6). Spirometry related features were generally scored higher in feature importance than other categories. However, it is important to note that most features have comparable levels of importance. Interestingly BMI scored a higher importance than both cardiovascular markers as well as oxygen saturation and respiratory rate, while other demographic markers were found to be of lesser importance.

### Example 9: Physicians Under-Estimate Dyspnea Severity

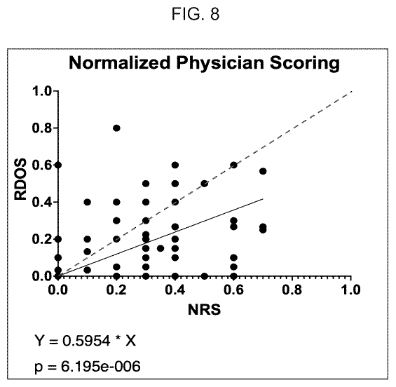

When RDOS were normalized to self-reported dyspnea severity scores, physicians significantly underestimate dyspnea severity (p=<0.001)(FIG. 8 and Table 4). When divided into a binary scoring system of identifying the presence of dyspnea, physician accuracy reached 56.86. The random forest model performed better than physicians on the dataset, reaching an accuracy of 84.09 for the current model. The largest improvements were in sensitivity (and thus naturally, false negative percentages were also much lower), as well as false positive. Thus, the model was better able to identify dyspnea severity within this population.

TABLE 4

| Predictor | Physicians | Algorithm |
|---|---|---|
| Sensitivity % | 36 | 76.67 |
| Specificity % | 76.92 | 86.27 |
| False Positive % | 23.08 | 13.73 |
| False Negative % | 64 | 23.33 |
| Accuracy % | 56.86 | 84.09 |

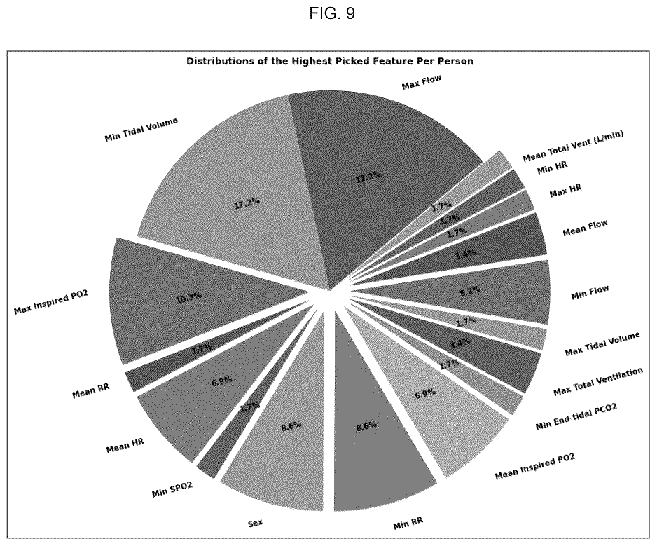

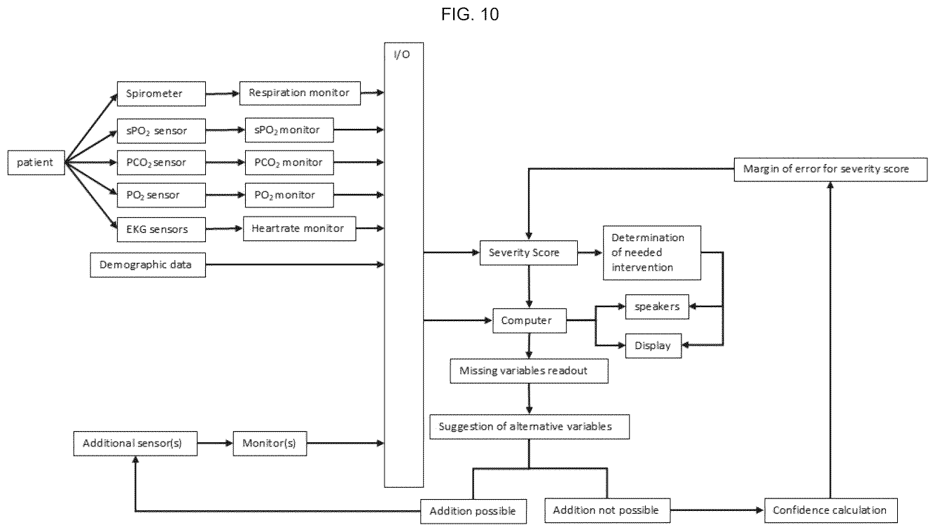

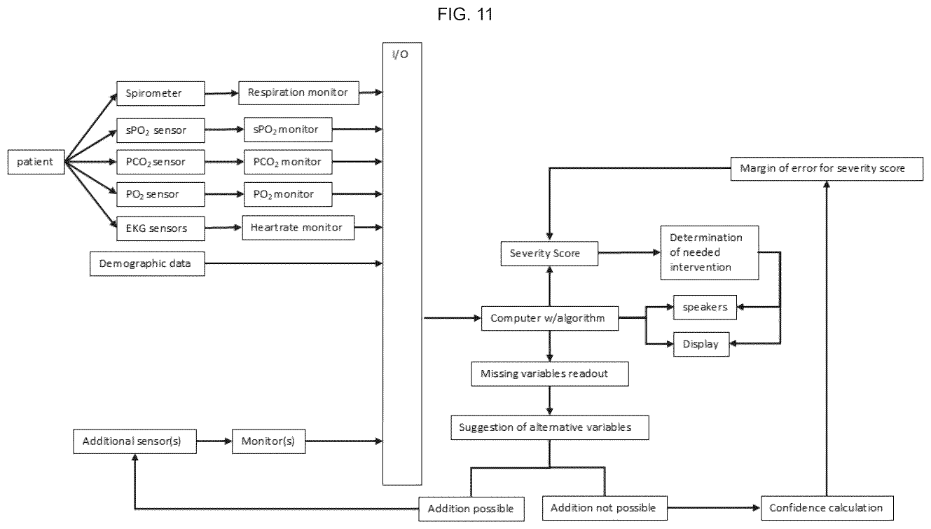

Participants displayed a large variance of highest rated feature importance. Each participant's data was individually run through the algorithm and the feature with the highest importance score was extracted. FIG. 9 shows the distribution of the highest scoring features. Spirometry-related features only accounted for roughly 50% of top features.

### Example 10: Discussion

Machine learning methods allow for the use of many variables to be considered simultaneously in a way that is difficult or impossible to do for unassisted humans. Also, the application of machine learning to monitoring technologies such as those for identifying dyspnea has key benefits, such as the ability to continuously monitor a patient. In this study, it was shown that using machine learning for a subjective sensation such as dyspnea is not only viable, but a superior method to professional observation alone. The model developed herein scored better in every performance metric compared to the physicians using observational scoring criteria. Furthermore, the application of machine learning to this dataset shows the huge variety of feature importance for each individual as they experience dyspnea. While the findings in this study and previous studies highlighting the significance of variables related to respiratory effort still hold true, there is also a plethora of other contributing variables which help to paint a better picture of the underlying mechanisms of dyspnea severity. A wide variety of data could have been added to the model, including further demographic data as well as information on the participant's psychological state, however these data types were excluded to focus on data that would be applicable to as many patients in the ICU as possible, and would require the least amount of human input as possible. Given the relationship between psychological factors such as anxiety and dyspnea, it can be hypothesized that anxiety would explain some portion of the individual variance in the data.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 markers refers to groups having 1, 2, or 3 markers. Similarly, a group having 1-5 markers refers to groups having 1, 2, 3, 4, or 5 markers, and so forth.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect disclosed herein can be used in combination with any other unless specifically indicated otherwise.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A computer-implemented method for determining the likelihood of a subject having dyspnea, the method comprising:
measuring in the subject at least 1, 2, 3, 4, 5, 6, 7, or 8 of: BMI, gender, age, pO₂, pCO₂, SpO₂, heart rate, and respiratory air flow; and
assigning a severity score based on the at least one measurement;
wherein the severity score exceeding a baseline value indicates the subject is likely to have dyspnea.

2. The method of claim 1, wherein the measured pCO₂ is mean end tidal pCO₂, and/or maximum end tidal pCO₂.

3. The method of claim 1, wherein the measured heart rate is mean heart rate and/or maximum heart rate.

4. The method of claim 1, wherein the measured respiratory air flow is at least one of: mean total ventilation, maximum total ventilation, mean tidal volume, maximum tidal volume, respiratory rate, mean flow, maximum flow, or any combination thereof.

5. The method of claim 1, wherein the measured SpO₂ is at least one of: mean SpO₂, maximum SpO₂, minimum SpO₂, or any combination thereof.

6. The method of claim 1, wherein the severity score is on a scale of 1 to 10, optionally wherein the score is: 0-2 indicating no dyspnea, 3-5 indicating moderate dyspnea, and 6-10 indicating severe dyspnea.

7. The method of claim 1, wherein at least BMI, end-tidal PCO₂, and SpO₂ are measured.

8. The method of claim 1, wherein the at least one measurements are repeated over a time period of at least every 1, 2, 3, 4, 5, 10, 20, 30, 60, 120, or 180 minutes.

9. A system for evaluating the severity of dyspnea in a subject, the system comprising:
a software system for measuring in the subject at least 1, 2, 3, 4, 5, 6, or 7 of: BMI, age, pO₂, pCO₂, SpO₂, heart rate, and respiratory air flow;
wherein the software system assigns a severity score based on the at least one measurement;
wherein a higher severity score is indicative of more severe dyspnea.

10. The system of claim 9, wherein the system further comprises at least 1, 2, 3, 4, 5, or 6 of: an air flow meter, respiration monitor, SpO₂ sensor, oxygen gas analyzer, carbon dioxide gas analyzer, electrocardiogram, or any combination thereof.

11. The system of claim 9, wherein the measured pCO₂ is mean end tidal pCO₂, and/or maximum end tidal pCO₂.

12. The system of claim 9, wherein the measured heart rate is mean heart rate and/or maximum heart rate.

13. The system of claim 9, wherein the measured respiratory air flow is at least one of: mean total ventilation, maximum total ventilation, mean tidal volume, maximum tidal volume, respiratory rate, mean flow, maximum flow, or any combination thereof.

14. The system of claim 9, wherein the measured SpO₂ is at least one of: mean SpO₂, maximum SpO₂, minimum SpO₂, or any combination thereof.

15. The system of claim 9, wherein the severity score is on a scale of 1 to 10, optionally wherein the score is: 0-2 indicating no dyspnea, 3-5 indicating moderate dyspnea, and 6-10 indicating severe dyspnea.

16. The system of claim 9, wherein at least BMI, end-tidal PCO₂, and SpO₂ are measured.

17. The system of claim 9, wherein the at least one measurements are repeated over a time period of at least every 1, 2, 3, 4, 5, 10, 20, 30, 60, 120, or 180 minutes.

18. A method of monitoring and treating dyspnea in a subject, the method comprising:
measuring in the subject at least 1, 2, 3, 4, 5, 6, 7, or 8 of: BMI, gender, age, pO₂, pCO₂, SpO₂, heart rate, and respiratory air flow;
assigning a severity score based on the at least one measurement; and
administering a treatment to the subject if the severity score is above a threshold value.

19. The method of claim 18, wherein the subject is mammalian and/or human.

20. The method of claim 18, wherein the treatment comprises a known therapy, procedure, pharmaceutical, or any combination thereof that is effective against dyspnea.

21. The method of claim 18, wherein the severity score is on a scale of 1 to 10, optionally wherein the score is: 0-2 indicating no dyspnea, 3-5 indicating moderate dyspnea, and 6-10 indicating severe dyspnea that requires administering treatment.
